(19) United States
(12) Patent Application Publication
(10) Pub. No.: US 2024/0379244 A1
(43) Pub. Date: Nov. 14, 2024

(54) AI-DRIVEN BIOMARKER BANK FOR LIVER LESION ANALYSIS

(71) Applicant: Siemens Healthcare GmbH, Erlangen (DE)

(72) Inventors: Zhoubing Xu, Plainsboro, NJ (US); Guillaume Chabin, Paris (FR); Alin Madalin Draghia, Brasov (RO); Sasa Grbic, Plainsboro, NJ (US)

(21) Appl. No.: 18/316,263

(22) Filed: May 12, 2023

(51) Int. Cl. G16H 50/70; G16H 30/20

(52) U.S. Cl. CPC G16H 50/70; G16H 30/20

(57) ABSTRACT

Systems and methods for performing an analysis on a patient population are provided. A biomarker bank storing lesion-related features extracted from medical images of a patient population is maintained. An analysis is performed on the patient population based on the lesion-related features stored in the biomarker bank. Results of the analysis.

### TECHNICAL FIELD

The present invention relates generally to AI (artificial intelligence)/ML (machine learning) for medical imaging analysis, and in particular to an AI-driven biomarker bank for liver lesion analysis.

### BACKGROUND

Liver cancer is cancer that starts in the liver of a patient and is one of the most lethal types of cancer. Early detection and characterization of liver cancer is important for subsequent treatment planning and positive patient prognosis. In the current clinical practice, liver lesions are manually detected and characterized by a radiologist from longitudinal imaging studies according to LI-RADS (liver reporting and data system) guidelines. However, such manual detection and characterization of liver lesions is a time consuming, labor intensive, and subjective task due to the complexity and variations of the liver anatomy and pathology, and do not consider characterizations of liver lesions of other patients. Recently, machine learning based approaches have been proposed for detection and classification of liver lesions. However, many analyses of liver lesions are performed according to user defined requirements, which cannot be represented with low-level latent features derived according to conventional machine learning based approaches.

### BRIEF SUMMARY OF THE INVENTION

In accordance with one or more embodiments, systems and methods for performing an analysis on a patient population are provided. A biomarker bank storing lesion-related features extracted from medical images of a patient population is maintained. An analysis is performed on the patient population based on the lesion-related features stored in the biomarker bank. Results of the analysis are output.

In one embodiment, the lesion-related features are in a standardized format for each lesion depicted in the medical images. The lesion-related features may be represented as a feature vector for each lesion depicted in the medical images.

In one embodiment, the biomarker bank is maintained by receiving the medical images of the patient population, extracting the lesion-related features from the medical images, and storing the lesion-related features in the biomarker bank.

In one embodiment, the lesion-related features comprise at least one of: measurement features, modality-specific features, patient-specific features, contrast phase differential features, and longitudinal differential features. In one embodiment, the lesion-related features comprise textures of the lesions for modalities of the medical images. In one embodiment, the lesion-related features comprise differentials of lesion intensities in the medical images along progression of contrast phases. In one embodiment, the lesion-related features comprise differentials between previously extracted lesion-related features.

In one embodiment, performing an analysis on the patient population comprise at least one of: computing a distribution for the patient population for one or more of the lesion-related features, identifying patients of the patient population with similar features, determining a treatment plan for a particular patient based on treatment plans of patients of the patient population identified as having similar features as the particular patient, automatically categorizing lesions according to LI-RADS (liver reporting and data systems) guidelines, generating standards relating to lesion malignancy, and generating a reporting schema describing lesions.

These and other advantages of the invention will be apparent to those of ordinary skill in the art by reference to the following detailed description and the accompanying drawings.

### BRIEF DESCRIPTION OF THE DRAWINGS

FIG. 1 shows a method for performing an analysis on a patient population, in accordance with one or more embodiments;

FIG. 2 shows a framework for performing an analysis on a patient population, in accordance with one or more embodiments;

FIG. 3 shows a method for updating a biomarker bank with lesion-related features, in accordance with one or more embodiments;

FIG. 4 shows an exemplary artificial neural network that may be used to implement one or more embodiments;

FIG. 5 shows a convolutional neural network that may be used to implement one or more embodiments; and

FIG. 6 shows a high-level block diagram of a computer that may be used to implement one or more embodiments.

### DETAILED DESCRIPTION

The present invention generally relates to methods and systems for an AI-driven biomarker bank for liver lesion analysis. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system.

Embodiments described herein provide for a biomarker bank for performing various analyses on a very large patient population. The biomarker bank is a computer database storing various types of biomarkers of a medical condition (e.g., liver cancer) for the patient population. The biomarkers are represented as lesion-related features extracted from medical images of the patient population. Advantageously, the biomarker bank may store a large number of different lesion-related features for the patient population to thereby enable performance of various exploratory analyses on the patient population. Such exploratory analyses are typically performed according to user defined requirements, which typically cannot be represented with low-level latent features derived from conventional machine learning based approaches. Further, the performance of such exploratory analyses on the patient population based on such a large number of lesion-related features of different types is not possible with conventional manual approaches, as users (e.g., radiologists) cannot practically perform such analyses on such a large patient population based on such a large number of biomarkers.

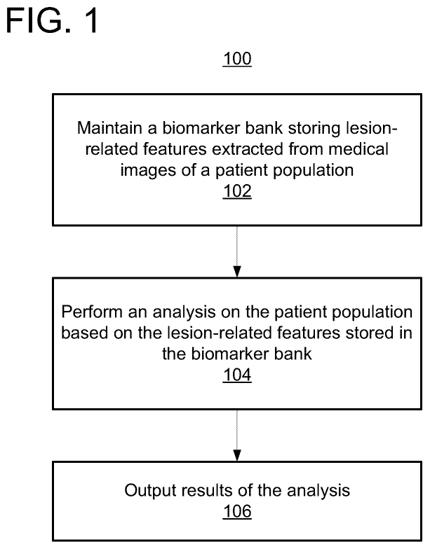

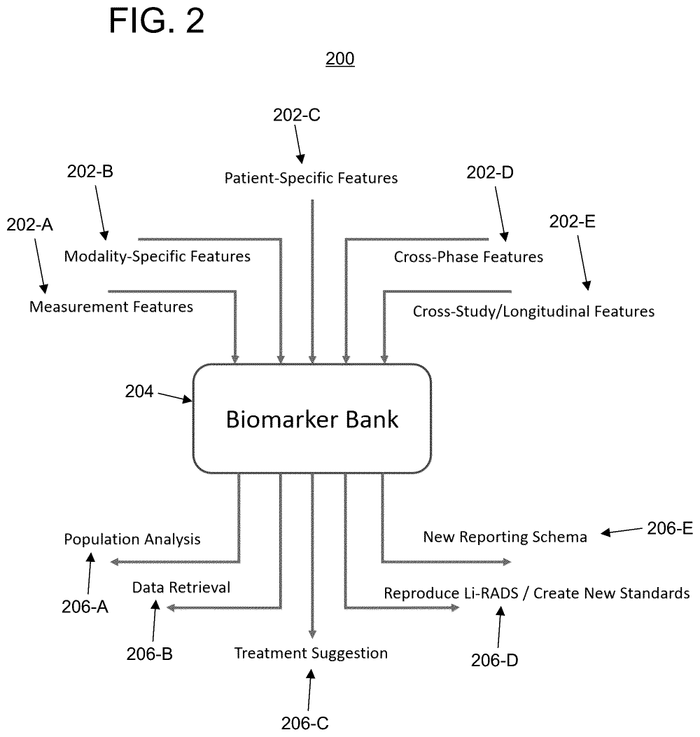

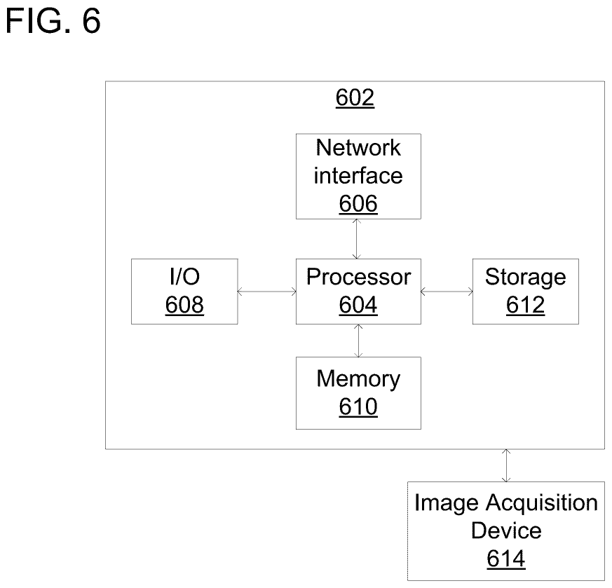

FIG. 1 shows a method 100 for performing an analysis on a patient population, in accordance with one or more embodiments. The steps of method 100 may be performed by one or more suitable computing devices, such as, e.g., computer 602 of FIG. 6. FIG. 2 shows a framework 200 for performing an analysis on a patient population, in accordance with one or more embodiments. FIG. 1 and FIG. 2 will be described together.

At step 102 of FIG. 1, a biomarker bank storing lesion-related features extracted from medical images of a patient population is maintained. The biomarker bank is a computer database implemented by one or more computing devices (computer 602 of FIG. 6) comprising memory or storage (e.g., memory 610 of FIG. 6) for storing the lesion-related features. In one example, as shown in framework 200 of FIG. 2, the biomarker bank is biomarker bank 204.

The lesion-related features may comprise one or more suitable features related to lesions depicted in the medical images. The lesions may comprise any injury or disease depicted in the medical images, such as, e.g., a wound, an ulcer, an abscess, a tumor, a nodule, or any other abnormality. The lesions may be located on an anatomical structure of a patient. In one embodiment, the anatomical structure is the liver of the patient. However, the anatomical structure may comprise any other organ, vessel, bone, or any other suitable anatomical structure of interest of the patient.

In one embodiment, as shown in FIG. 2, the lesion-related features may comprise measurement features 202-A, modality-specific features 202-B, patient-specific features 202-C, cross-phase features 202-D, and cross-study/longitudinal features 202-E stored in biomarker bank 204. Measurement features 202-A comprise any measurement of lesions depicted in the medical images, such as, e.g., lesion diameter, lesion volume size, lesion area per slice, etc. Modality-specific features 202-B comprise features of the lesions specific to the modality of the medical images. In one example, modality-specific features 202-B comprise a texture of the lesions for each modality of the medical images. The texture may be extracted, for example, along image-grids (which may be useful for differentiating normal parenchyma from fibrosis, cirrhosis, and cancerous tissues) or along the centroid to the peripheral direction for each lesion (which may be useful for recognizing capsule patterns). Patient-specific features 202-C comprise features of anatomical features of the patient that are in proximity to the lesions, such as, e.g., the volume of the anatomical structure (e.g., the liver or other organs) on which the lesions are located or in proximity to the lesions, the parenchyma density of the anatomical structure on which the lesions are located or in proximity to the lesions, or other relevant anatomical features specific to the patient. Cross contrast phase features 202-D comprise differentials of lesion intensities in the medical images along the progression of contrast phases of a contrast agent (e.g., pre-contrast, early arterial phase, later arterial phase, portal venous phase, nephrogenic phase, excretory phase). The differentials of lesion intensities can be normalized by the time intervals between contrast phases. Cross-study/longitudinal features 202-E comprise differentials between previously extracted lesion-related features from the medical images in longitudinal studies. In one example, cross-study/longitudinal features 202-E comprise differentials between lesion size or any other of measurement features 202-A, modality-specific features 202-B, or patient-specific features 202-C. The differentials between previously extracted lesion-related features can be normalized by the time intervals between longitudinal studies.

The lesion-related features may be automatically or semi-automatically extracted from the medical images using AI (artificial intelligence)/ML (machine learning) based algorithms. For example, such AI/ML based algorithms may comprise known AI/ML based algorithms for detection, classification, segmentation, quantification, or any other medical imaging analysis task. In one example, the lesion-related features are extracted from the medical images according to the methods described in U.S. patent application Ser. No. 17/648,940, titled “AI Driven Longitudinal Liver Focal Lesion Analysis,” the disclosure of which is herein incorporated by reference in its entirety.

The lesion-related features may be stored in the biomarker bank according to a standardized format for each lesion depicted in the medical images. In one embodiment, the lesion-related features are stored as a respective feature vector for each lesion depicted in the medical images. The format of the feature vectors is standardized across the lesions. Each feature vector is associated with one specific lesion entity and has a fixed length of N that comprises features of multiple types. Each of the types of features correspond to the same location within the feature vectors. For example, features derived from an intensity histogram of a lesion on an MR (magnetic resonance) T1 pre-contrast sequence may be located from index 0 to index 15 in the feature vectors, features derived from an MR T1 arterial phase may be located from index 16 to index 31 in the feature vectors, the difference of intensity between T1 pre-contrast and T1 arterial phase may be located from index 32 to index 47, etc. In some embodiments, the feature vectors may comprise blank portions, for example, where a particular imaging modality is not available.

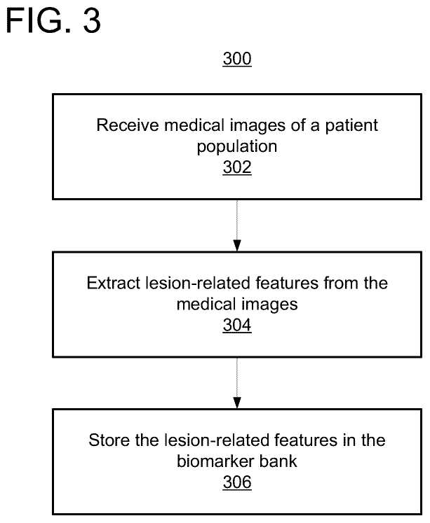

In one embodiment, the biomarker bank is maintained by performing one or more tasks to ensure functionality of the biomarker bank. For example, such tasks may include backing up data, checking data integrity, etc. In another embodiment, the biomarker bank is maintained by updating the biomarker bank with lesion-related features, as described in further detail below with respect to FIG. 3.

FIG. 3 shows a method 300 for updating a biomarker bank with lesion-related features, in accordance with one or more embodiments. The steps of method 300 may be performed by one or more suitable computing devices, such as, e.g., computer 602 of FIG. 6. The steps of method 300 may be performed at step 102 of FIG. 1.

At step 302 of FIG. 3, medical images of a patient population are received. The medical images received at step 302 of FIG. 3 may be the medical images at step 102 of FIG. 1. The medical images may depict one or more lesions located on an anatomical structure of a patient. The medical images may be of any suitable modality, such as, e.g., CT (computed tomography), MRI (magnetic resonance imaging), ultrasound, x-ray, or any other medical imaging modality or combinations of medical imaging modalities. The first medical images may be 2D (two dimensional) images and/or 3D (three dimensional) volumes. Accordingly, reference herein to pixels of a 2D image equally refer to voxels of a 3D volume. The medical images may be received directly from an image acquisition device, such as, e.g., a CT scanner, as the medical images are acquired, may be received by loading previously acquired medical images from a storage or memory of a computer system, or may be received from a remote computer system.

At step 304 of FIG. 3, lesion-related features are extracted from the medical images. The lesion-related features extracted at step 304 of FIG. 3 may be the lesion-related features at step 102 of FIG. 1. The lesion-related features may be extracted as discussed above with respect to step 102 of FIG. 1. However, the lesion-related features may be extracted according to any suitable approach.

At step 306 of FIG. 3, the lesion-related features are stored in the biomarker bank. For example, the lesion-related features may be stored on memory or storage of a computer database implementing the biomarker bank or by transmitting the lesion-related features to a remote computer data implementing the biomarker bank for storage on memory or storage.

Referring back to FIG. 1, at step 104, an analysis on the patient population is performed based on the lesion-related features stored in the biomarker bank. The analysis may comprise any suitable analysis on the patient population.

In one example, as shown in framework 200 of FIG. 2, the analysis of the patient population may comprise one or more of population analysis 206-A, data retrieval 206-B, treatment suggestion 206-C, reproduce LI-RADS (liver reporting and data systems)/create new standards 206-D, or new reporting schema 206-E. Population analysis 206-A may comprise computing a distribution for the patient population for one or more of the lesion-related features. Data retrieval 206-B may comprise identifying patients of the patient population with similar features of the lesion-related features. Treatment suggestion 206-C may comprise determining a treatment plan for a particular patient based on treatment plans of patients of the patient population identified as having similar features as the particular patient. Reproduce LI-RADS/create new standards 206-D may comprise automatically categorizing lesions according to LI-RADS guidelines or generating quantitative, objective, and consistent standards by exploring the lesion-related features that most relate to lesion malignancy using biopsy results as ground truth. New reporting schema 206-E may comprise directly using the biomarker bank as the code to describe the liver lesion (if the standards created at 206-D are well accepted and proven highly correlated with lesion malignancy).

In one exemplary analysis of the patient population, during a treatment planning period, given a target lesion of a new patient, a feature vector may be generated for the target lesion according to the standardized format. The newly generated feature vector can be compared with one or more feature vectors generated from previously analyzed lesions and maintained in the biomarker bank. In one embodiment, the comparison may be performed by computing pair-wise distances between the feature vector generated for the target lesion and the one or more feature vectors maintained in the biomarker bank. In another embodiment, in a more computationally efficient approach, the comparison may be performed by projecting the feature vector for the target lesion and the one or more feature vectors maintained in the biomarker bank to a pre-built low-dimensional space and computing the pair-wise distance between the target lesion and the one or more feature vectors maintained in the biomarker bank in the low-dimensional space. Based on the comparison, a user (e.g., clinicians) may determine the k nearest feature vectors to the feature vector for the target lesion. Each retrieved feature vector is associated with a previously diagnosed or treated lesion, and therefore the treatment outcome can be also retrieved. If the previous treatment outcomes for a similar lesion indicates that the treatment approach was effective, then the clinicians may implement a similar treatment approach to the new lesion. Otherwise, the clinicians may implement an alternative treatment approach other than the treatment approach that was not effective.

At step 106 of FIG. 1, results of the analysis are output. For example, the results of the analysis can be output by displaying the results of the analysis on a display device of a computer system, storing the results of the analysis on a memory or storage of a computer system, or by transmitting the results of the analysis to a remote computer system.

Embodiments described herein are described with respect to the claimed systems as well as with respect to the claimed methods. Features, advantages or alternative embodiments herein can be assigned to the other claimed objects and vice versa. In other words, claims for the systems can be improved with features described or claimed in the context of the methods. In this case, the functional features of the method are embodied by objective units of the providing system.

Furthermore, certain embodiments described herein are described with respect to methods and systems utilizing trained machine learning based models, as well as with respect to methods and systems for training machine learning based models. Features, advantages or alternative embodiments herein can be assigned to the other claimed objects and vice versa. In other words, claims for methods and systems for training a machine learning based model can be improved with features described or claimed in context of the methods and systems for utilizing a trained machine learning based model, and vice versa.

In particular, the trained machine learning based models applied in embodiments described herein can be adapted by the methods and systems for training the machine learning based models. Furthermore, the input data of the trained machine learning based model can comprise advantageous features and embodiments of the training input data, and vice versa. Furthermore, the output data of the trained machine learning based model can comprise advantageous features and embodiments of the output training data, and vice versa.

In general, a trained machine learning based model mimics cognitive functions that humans associate with other human minds. In particular, by training based on training data, the trained machine learning based model is able to adapt to new circumstances and to detect and extrapolate patterns.

In general, parameters of a machine learning based model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the trained machine learning based model can be adapted iteratively by several steps of training.

In particular, a trained machine learning based model can comprise a neural network, a support vector machine, a decision tree, and/or a Bayesian network, and/or the trained machine learning based model can be based on k-means clustering, Q-learning, genetic algorithms, and/or association rules. In particular, a neural network can be a deep neural network, a convolutional neural network, or a convolutional deep neural network. Furthermore, a neural network can be an adversarial network, a deep adversarial network and/or a generative adversarial network.

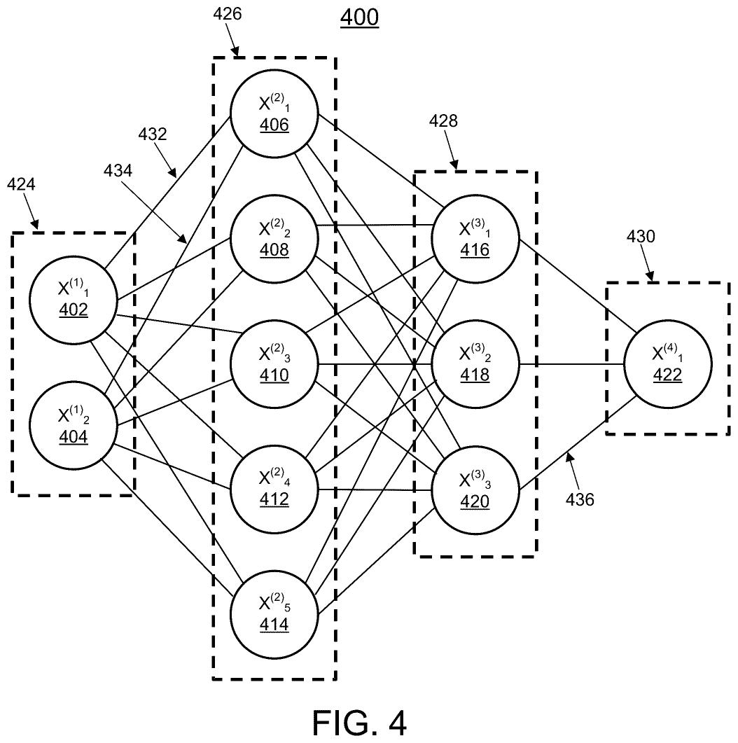

FIG. 4 shows an embodiment of an artificial neural network 400, in accordance with one or more embodiments. Alternative terms for “artificial neural network” are “neural network”, “artificial neural net” or “neural net”. Machine learning networks described herein, such as, e.g., the machine learning based networks utilized for extracting the lesion-related features from the medical images described with respect to step 102 of FIG. 1 or the machine learning based networks utilized for performing step 304 of FIG. 3, may be implemented using artificial neural network 400.

The artificial neural network 400 comprises nodes 402-422 and edges 432, 434, . . . , 436, wherein each edge 432, 434, . . . , 436 is a directed connection from a first node 402-422 to a second node 402-422. In general, the first node 402-422 and the second node 402-422 are different nodes 402-422, it is also possible that the first node 402-422 and the second node 402-422 are identical. For example, in FIG. 4, the edge 432 is a directed connection from the node 402 to the node 406, and the edge 434 is a directed connection from the node 404 to the node 406. An edge 432, 434, . . . , 436 from a first node 402-422 to a second node 402-422 is also denoted as “ingoing edge” for the second node 402-422 and as “outgoing edge” for the first node 402-422.

In this embodiment, the nodes 402-422 of the artificial neural network 400 can be arranged in layers 424-430, wherein the layers can comprise an intrinsic order introduced by the edges 432, 434, . . . , 436 between the nodes 402-422. In particular, edges 432, 434, . . . , 436 can exist only between neighboring layers of nodes. In the embodiment shown in FIG. 4, there is an input layer 424 comprising only nodes 402 and 404 without an incoming edge, an output layer 430 comprising only node 422 without outgoing edges, and hidden layers 426, 428 in-between the input layer 424 and the output layer 430. In general, the number of hidden layers 426, 428 can be chosen arbitrarily. The number of nodes 402 and 404 within the input layer 424 usually relates to the number of input values of the neural network 400, and the number of nodes 422 within the output layer 430 usually relates to the number of output values of the neural network 400.

In particular, a (real) number can be assigned as a value to every node 402-422 of the neural network 400. Here, x⁽ⁿ⁾ᵢ denotes the value of the i-th node 402-422 of the n-th layer 424-430. The values of the nodes 402-422 of the input layer 424 are equivalent to the input values of the neural network 400, the value of the node 422 of the output layer 430 is equivalent to the output value of the neural network 400. Furthermore, each edge 432, 434, . . . , 436 can comprise a weight being a real number, in particular, the weight is a real number within the interval [−1, 1] or within the interval [0, 1]. Here, w^{(m,n)}_{i,j} denotes the weight of the edge between the i-th node 402-422 of the m-th layer 424-430 and the j-th node 402-422 of the n-th layer 424-430. Furthermore, the abbreviation w^{(m,n)}_{i,j} is defined for the weight w^{(n,n+1)}_{i,j}.

In particular, to calculate the output values of the neural network 400, the input values are propagated through the neural network. In particular, the values of the nodes 402-422 of the (n+1)-th layer 424-430 can be calculated based on the values of the nodes 402-422 of the n-th layer 424-430 by

$${x}_{j}^{( n + 1 )} = f ( {\sum }_{i} {x}_{i}^{( n )} \cdot {w}_{i , j}^{( n )} ) .$$

Herein, the function f is a transfer function (another term is “activation function”). Known transfer functions are step functions, sigmoid function (e.g. the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 424 are given by the input of the neural network 400, wherein values of the first hidden layer 426 can be calculated based on the values of the input layer 424 of the neural network, wherein values of the second hidden layer 428 can be calculated based in the values of the first hidden layer 426, etc.

In order to set the values w^{(m,n)}_{i,j} for the edges, the neural network 400 has to be trained using training data. In particular, training data comprises training input data and training output data (denoted as tᵢ). For a training step, the neural network 400 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 400 (backpropagation algorithm). In particular, the weights are changed according to

$${δ}_{j}^{( n )} = ( {x}_{k}^{( n + 1 )} - {t}_{j}^{( n + 1 )} ) \cdot {f}^{′} ( {\sum }_{i} {x}_{i}^{( n )} \cdot {w}_{i , j}^{( n )} )$$

wherein γ is a learning rate, and the numbers δ⁽ⁿ⁾ⱼ can be recursively calculated as

$${w}_{i , j}^{′ ( n )} = {w}_{i , j}^{( n )} - γ \cdot {δ}_{j}^{( n )} \cdot {x}_{i}^{( n )}$$

based on δ⁽ⁿ⁺¹⁾ⱼ, if the (n+1)-th layer is not the output layer, and

$${δ}_{j}^{( n )} = ( {\sum }_{k} {δ}_{k}^{( n + 1 )} \cdot {w}_{j , k}^{( n + 1 )} ) \cdot {f}^{′} ( {\sum }_{i} {x}_{i}^{( n )} \cdot {w}_{i , j}^{( n )} )$$

if the (n+1)-th layer is the output layer 430, wherein f′ is the first derivative of the activation function, and y⁽ⁿ⁺¹⁾ⱼ is the comparison training value for the j-th node of the output layer 430.

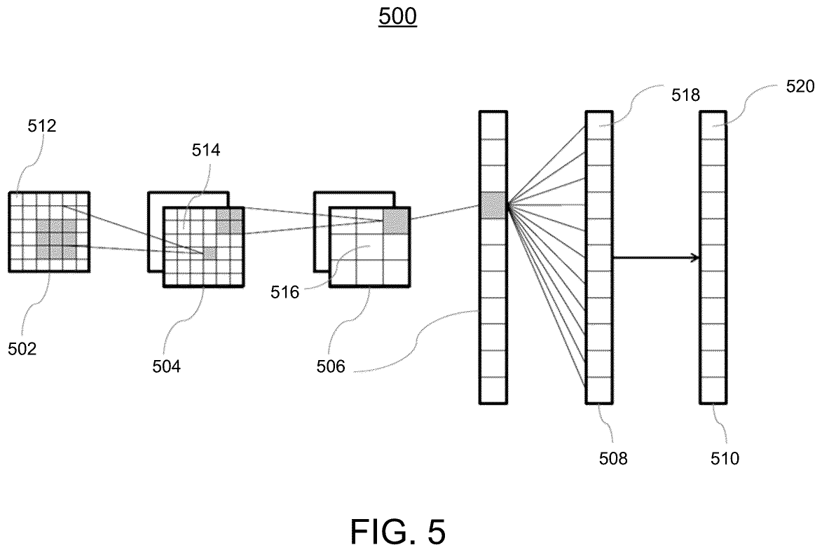

FIG. 5 shows a convolutional neural network 500, in accordance with one or more embodiments. Machine learning networks described herein, such as, e.g., the machine learning based networks utilized for extracting the lesion-related features from the medical images described with respect to step 102 of FIG. 1 or the machine learning based networks utilized for performing step 304 of FIG. 3, may be implemented using convolutional neural network 500.

In the embodiment shown in FIG. 5, the convolutional neural network comprises 500 an input layer 502, a convolutional layer 504, a pooling layer 506, a fully connected layer 508, and an output layer 510. Alternatively, the convolutional neural network 500 can comprise several convolutional layers 504, several pooling layers 506, and several fully connected layers 508, as well as other types of layers. The order of the layers can be chosen arbitrarily, usually fully connected layers 508 are used as the last layers before the output layer 510.

In particular, within a convolutional neural network 500, the nodes 512-520 of one layer 502-510 can be considered to be arranged as a d-dimensional matrix or as a d-dimensional image. In particular, in the two-dimensional case the value of the node 512-520 indexed with i and j in the n-th layer 502-510 can be denoted as x⁽ⁿ⁾_{[i,j]}. However, the arrangement of the nodes 512-520 of one layer 502-510 does not have an effect on the calculations executed within the convolutional neural network 500 as such, since these are given solely by the structure and the weights of the edges.

In particular, a convolutional layer 504 is characterized by the structure and the weights of the incoming edges forming a convolution operation based on a certain number of kernels. In particular, the structure and the weights of the incoming edges are chosen such that the values x⁽ⁿ⁾ₖ of the nodes 514 of the convolutional layer 504 are calculated as a convolution x⁽ⁿ⁾ₖ=Kₖ*x⁽ⁿ⁻¹⁾ based on the values x⁽ⁿ⁻¹⁾ of the nodes 512 of the preceding layer 502, where the convolution * is defined in the two-dimensional case as

$${x}_{k}^{( n )} [ i , j ] = ( {K}_{k} * {x}^{( n - 1 )} ) [ i , j ] = {\sum }_{{i}^{′}} {\sum }_{{j}^{′}} {K}_{k} [ {i}^{′} , {j}^{′} ] \cdot {x}^{( n - 1 )} [ i - {i}^{′} , j - {j}^{′} ] .$$

Here the k-th kernel Kₖ is a d-dimensional matrix (in this embodiment a two-dimensional matrix), which is usually small compared to the number of nodes 512-518 (e.g. a 3×3 matrix, or a 5×5 matrix). In particular, this implies that the weights of the incoming edges are not independent, but chosen such that they produce said convolution equation. In particular, for a kernel being a 3×3 matrix, there are only 9 independent weights (each entry of the kernel matrix corresponding to one independent weight), irrespectively of the number of nodes 512-520 in the respective layer 502-510. In particular, for a convolutional layer 504, the number of nodes 514 in the convolutional layer is equivalent to the number of nodes 512 in the preceding layer 502 multiplied with the number of kernels.

If the nodes 512 of the preceding layer 502 are arranged as a d-dimensional matrix, using a plurality of kernels can be interpreted as adding a further dimension (denoted as “depth” dimension), so that the nodes 514 of the convolutional layer 504 are arranged as a (d+1)-dimensional matrix. If the nodes 512 of the preceding layer 502 are already arranged as a (d+1)-dimensional matrix comprising a depth dimension, using a plurality of kernels can be interpreted as expanding along the depth dimension, so that the nodes 514 of the convolutional layer 504 are arranged also as a (d+1)-dimensional matrix, wherein the size of the (d+1)-dimensional matrix with respect to the depth dimension is by a factor of the number of kernels larger than in the preceding layer 502.

The advantage of using convolutional layers 504 is that spatially local correlation of the input data can exploited by enforcing a local connectivity pattern between nodes of adjacent layers, in particular by each node being connected to only a small region of the nodes of the preceding layer.

In embodiment shown in FIG. 5, the input layer 502 comprises 36 nodes 512, arranged as a two-dimensional 6×6 matrix. The convolutional layer 504 comprises 72 nodes 514, arranged as two two-dimensional 6×6 matrices, each of the two matrices being the result of a convolution of the values of the input layer with a kernel. Equivalently, the nodes 514 of the convolutional layer 504 can be interpreted as arranges as a three-dimensional 6×6×2 matrix, wherein the last dimension is the depth dimension.

A pooling layer 506 can be characterized by the structure and the weights of the incoming edges and the activation function of its nodes 516 forming a pooling operation based on a non-linear pooling function f. For example, in the two dimensional case the values x⁽ⁿ⁾ of the nodes 516 of the pooling layer 506 can be calculated based on the values x⁽ⁿ⁻¹⁾ of the nodes 514 of the preceding layer 504 as

$${x}^{( n )} [ i , j ] = f ( {x}^{( n - 1 )} [ {id}_{1} , {jd}_{2} ] , ... , {x}^{( n - 1 )} [ {id}_{1} + {d}_{1} - 1 , {jd}_{2} + {d}_{2} - 1 ] )$$

In other words, by using a pooling layer 506, the number of nodes 514, 516 can be reduced, by replacing a number d1·d2 of neighboring nodes 514 in the preceding layer 504 with a single node 516 being calculated as a function of the values of said number of neighboring nodes in the pooling layer. In particular, the pooling function f can be the max-function, the average or the L2-Norm. In particular, for a pooling layer 506 the weights of the incoming edges are fixed and are not modified by training.

The advantage of using a pooling layer 506 is that the number of nodes 514, 516 and the number of parameters is reduced. This leads to the amount of computation in the network being reduced and to a control of overfitting.

In the embodiment shown in FIG. 5, the pooling layer 506 is a max-pooling, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

A fully-connected layer 508 can be characterized by the fact that a majority, in particular, all edges between nodes 516 of the previous layer 506 and the nodes 518 of the fully-connected layer 508 are present, and wherein the weight of each of the edges can be adjusted individually.

In this embodiment, the nodes 516 of the preceding layer 506 of the fully-connected layer 508 are displayed both as two-dimensional matrices, and additionally as non-related nodes (indicated as a line of nodes, wherein the number of nodes was reduced for a better presentability). In this embodiment, the number of nodes 518 in the fully connected layer 508 is equal to the number of nodes 516 in the preceding layer 506. Alternatively, the number of nodes 516, 518 can differ.

Furthermore, in this embodiment, the values of the nodes 520 of the output layer 510 are determined by applying the Softmax function onto the values of the nodes 518 of the preceding layer 508. By applying the Softmax function, the sum the values of all nodes 520 of the output layer 510 is 1, and all values of all nodes 520 of the output layer are real numbers between 0 and 1.

A convolutional neural network 500 can also comprise a ReLU (rectified linear units) layer or activation layers with non-linear transfer functions. In particular, the number of nodes and the structure of the nodes contained in a ReLU layer is equivalent to the number of nodes and the structure of the nodes contained in the preceding layer. In particular, the value of each node in the ReLU layer is calculated by applying a rectifying function to the value of the corresponding node of the preceding layer.

The input and output of different convolutional neural network blocks can be wired using summation (residual/dense neural networks), element-wise multiplication (attention) or other differentiable operators. Therefore, the convolutional neural network architecture can be nested rather than being sequential if the whole pipeline is differentiable.

In particular, convolutional neural networks 500 can be trained based on the backpropagation algorithm. For preventing overfitting, methods of regularization can be used, e.g. dropout of nodes 512-520, stochastic pooling, use of artificial data, weight decay based on the L1 or the L2 norm, or max norm constraints. Different loss functions can be combined for training the same neural network to reflect the joint training objectives. A subset of the neural network parameters can be excluded from optimization to retain the weights pretrained on another datasets.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatus, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 1 or 3. Certain steps or functions of the methods and workflows described herein, including one or more of the steps or functions of FIG. 1 or 3, may be performed by a server or by another processor in a network-based cloud-computing system. Certain steps or functions of the methods and workflows described herein, including one or more of the steps of FIG. 1 or 3, may be performed by a client computer in a network-based cloud computing system. The steps or functions of the methods and workflows described herein, including one or more of the steps of FIG. 1 or 3, may be performed by a server and/or by a client computer in a network-based cloud computing system, in any combination.

Systems, apparatus, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 1 or 3, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 602 that may be used to implement systems, apparatus, and methods described herein is depicted in FIG. 6. Computer 602 includes a processor 604 operatively coupled to a data storage device 612 and a memory 610. Processor 604 controls the overall operation of computer 602 by executing computer program instructions that define such operations. The computer program instructions may be stored in data storage device 612, or other computer readable medium, and loaded into memory 610 when execution of the computer program instructions is desired. Thus, the method and workflow steps or functions of FIG. 1 or 3 can be defined by the computer program instructions stored in memory 610 and/or data storage device 612 and controlled by processor 604 executing the computer program instructions. For example, the computer program instructions can be implemented as computer executable code programmed by one skilled in the art to perform the method and workflow steps or functions of FIG. 1 or 3. Accordingly, by executing the computer program instructions, the processor 604 executes the method and workflow steps or functions of FIG. 1 or 3. Computer 602 may also include one or more network interfaces 606 for communicating with other devices via a network. Computer 602 may also include one or more input/output devices 608 that enable user interaction with computer 602 (e.g., display, keyboard, mouse, speakers, buttons, etc.).

Processor 604 may include both general and special purpose microprocessors, and may be the sole processor or one of multiple processors of computer 602. Processor 604 may include one or more central processing units (CPUs), for example. Processor 604, data storage device 612, and/or memory 610 may include, be supplemented by, or incorporated in, one or more application-specific integrated circuits (ASICs) and/or one or more field programmable gate arrays (FPGAs).

Data storage device 612 and memory 610 each include a tangible non-transitory computer readable storage medium. Data storage device 612, and memory 610, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 608 may include peripherals, such as a printer, scanner, display screen, etc. For example, input/output devices 608 may include a display device such as a cathode ray tube (CRT) or liquid crystal display (LCD) monitor for displaying information to the user, a keyboard, and a pointing device such as a mouse or a trackball by which the user can provide input to computer 602.

An image acquisition device 614 can be connected to the computer 602 to input image data (e.g., medical images) to the computer 602. It is possible to implement the image acquisition device 614 and the computer 602 as one device. It is also possible that the image acquisition device 614 and the computer 602 communicate wirelessly through a network. In a possible embodiment, the computer 602 can be located remotely with respect to the image acquisition device 614.

Any or all of the systems and apparatus discussed herein may be implemented using one or more computers such as computer 602.

One skilled in the art will recognize that an implementation of an actual computer or computer system may have other structures and may contain other components as well, and that FIG. 6 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A computer-implemented method comprising:
maintaining a biomarker bank storing lesion-related features extracted from medical images of a patient population;
performing an analysis on the patient population based on the lesion-related features stored in the biomarker bank; and
outputting results of the analysis.

2. The computer-implemented method of claim 1, wherein the lesion-related features are in a standardized format for each lesion depicted in the medical images.

3. The computer-implemented method of claim 1, wherein the lesion-related features are represented as a feature vector for each lesion depicted in the medical images.

4. The computer-implemented method of claim 1, wherein maintaining a biomarker bank storing lesion-related features extracted from medical images of a patient population comprises:
receiving the medical images of the patient population;
extracting the lesion-related features from the medical images; and
storing the lesion-related features in the biomarker bank.

5. The computer-implemented method of claim 1, wherein the lesion-related features comprise at least one of: measurement features, modality-specific features, patient-specific features, contrast phase differential features, and longitudinal differential features.

6. The computer-implemented method of claim 1, wherein the lesion-related features comprise textures of the lesions for modalities of the medical images.

7. The computer-implemented method of claim 1, wherein the lesion-related features comprise differentials of lesion intensities in the medical images along progression of contrast phases.

8. The computer-implemented method of claim 1, wherein the lesion-related features comprise differentials between previously extracted lesion-related features.

9. The computer-implemented method of claim 1, wherein performing an analysis on the patient population based on the lesion-related features stored in the biomarker bank comprises at least one of:
computing a distribution for the patient population for one or more of the lesion-related features,
identifying patients of the patient population with similar features,
determining a treatment plan for a particular patient based on treatment plans of patients of the patient population identified as having similar features as the particular patient,
automatically categorizing lesions according to LI-RADS (liver reporting and data systems) guidelines,
generating standards relating to lesion malignancy, and
generating a reporting schema describing lesions.

10. An apparatus comprising:
means for maintaining a biomarker bank storing lesion-related features extracted from medical images of a patient population;
means for performing an analysis on the patient population based on the lesion-related features stored in the biomarker bank; and
means for outputting results of the analysis.

11. The apparatus of claim 10, wherein the lesion-related features are in a standardized format for each lesion depicted in the medical images.

12. The apparatus of claim 10, wherein the lesion-related features are represented as a feature vector for each lesion depicted in the medical images.

13. The apparatus of claim 10, wherein the means for maintaining a biomarker bank storing lesion-related features extracted from medical images of a patient population comprises:
means for receiving the medical images of the patient population;
means for extracting the lesion-related features from the medical images; and
means for storing the lesion-related features in the biomarker bank.

14. The apparatus of claim 10, wherein the lesion-related features comprise at least one of: measurement features, modality-specific features, patient-specific features, contrast phase differential features, and longitudinal differential features.

15. A non-transitory computer readable medium storing computer program instructions, the computer program instructions when executed by a processor cause the processor to perform operations comprising:
maintaining a biomarker bank storing lesion-related features extracted from medical images of a patient population;
performing an analysis on the patient population based on the lesion-related features stored in the biomarker bank; and
outputting results of the analysis.

16. The non-transitory computer readable medium of claim 15, wherein the lesion-related features are in a standardized format for each lesion depicted in the medical images.

17. The non-transitory computer readable medium of claim 15, wherein the lesion-related features comprise textures of the lesions for modalities of the medical images.

18. The non-transitory computer readable medium of claim 15, wherein the lesion-related features comprise differentials of lesion intensities in the medical images along progression of contrast phases.

19. The non-transitory computer readable medium of claim 15, wherein the lesion-related features comprise differentials between previously extracted lesion-related features.

20. The non-transitory computer readable medium of claim 15, wherein performing an analysis on the patient population based on the lesion-related features stored in the biomarker bank comprises at least one of:
computing a distribution for the patient population for one or more of the lesion-related features,
identifying patients of the patient population with similar features,
determining a treatment plan for a particular patient based on treatment plans of patients of the patient population identified as having similar features as the particular patient,
automatically categorizing lesions according to LI-RADS (liver reporting and data systems) guidelines,
generating standards relating to lesion malignancy, and
generating a reporting schema describing lesions.
